SEQUENCE LISTING (10 sequences)

SEQ ID NO: 1 - DNA, 20 bp, synthetic construct
  source 1..20: mol_type = other DNA
agctacgaatctccgaccac

SEQ ID NO: 2 - DNA, 22 bp, synthetic construct
  source 1..22: mol_type = other DNA
cgttatcccatgtgtcgaagaa

SEQ ID NO: 3 - DNA, 23 bp, synthetic construct
  source 1..23: mol_type = other DNA
actgagagtgattgagagtggac

SEQ ID NO: 4 - DNA, 21 bp, synthetic construct
  source 1..21: mol_type = other DNA
aaccctctgcacccagttttc

SEQ ID NO: 5 - DNA, 21 bp, synthetic construct
  source 1..21: mol_type = other DNA
cctgaaccttccaaagatggc

SEQ ID NO: 6 - DNA, 21 bp, synthetic construct
  source 1..21: mol_type = other DNA
ttcaccaggcaagtctcctca

SEQ ID NO: 7 - DNA, 20 bp, synthetic construct
  source 1..20: mol_type = other DNA
tcaaggcgcatgtgaactcc

SEQ ID NO: 8 - DNA, 22 bp, synthetic construct
  source 1..22: mol_type = other DNA
gatgtcaaactcactcatggct

SEQ ID NO: 9 - DNA, 21 bp, synthetic construct
  source 1..21: mol_type = other DNA
tgcatcgttttgggttctctt

SEQ ID NO: 10 - DNA, 27 bp, synthetic construct
  source 1..27: mol_type = other DNA
agtttgaagtaaaaggagacaatttgg

(19) United States
(12) Patent Application Publication
(10) Pub. No.: US 2024/0245746 A1
(43) Pub. Date: Jul. 25, 2024

(54) METHOD FOR REGULATING IMMUNITY BY USING BLACK HULLESS BARLEY EXTRACT

(71) Applicant: TCI CO., LTD., Taipei (TW)

(72) Inventors: YUNG-HSIANG LIN, Taipei (TW); Pei-Han CHANG CHIEN, Taipei (TW)

(21) Appl. No.: 18/518,451

(22) Filed: Nov. 23, 2023

Related U.S. Application Data
(60) Provisional application No. 63/480,512, filed on Jan. 19, 2023.

(51) Int. Cl. A61K 36/8998; A61P 11/14; A61P 25/04; A61P 29/00

(52) U.S. Cl. CPC A61K 36/8998; A61P 11/14; A61P 25/04; A61P 29/00; A61K 2236/19

(57) ABSTRACT

A method for regulating immunity is provided. The method includes administering to a subject in need thereof a composition that includes an effective dose of black hulless barley extract. The black hulless barley extract is obtained by extracting seeds of black hulless barley (Hordeum vulgare L. var. nudum Hook. f.) with an amylase aqueous solution.

### CROSS-REFERENCE TO RELATED APPLICATION

This application claims the benefit of U.S. provisional application Ser. No. 63/480,512, filed on Jan. 19, 2023. The entirety of the above-mentioned patent application is hereby incorporated by reference herein and made a part of the specification.

### REFERENCE OF AN ELECTRONIC SEQUENCE LISTING

The contents of the electronic sequence listing (P234134USI.xml; Size: 10,005 bytes; and Date of Creation: Nov. 21, 2023) is herein incorporated by reference in its entirety.

### BACKGROUND

### Technical Field

The present disclosure relates to a black hulless barley extract, and in particular to a black hulless barley extract prepared by extraction with an amylase aqueous solution, which has a function of regulating immunity.

### Related Art

Hulless barley, also known as naked barley, highland barley and pearl barley, is a cereal crop and one of the varieties of barley. On the Qinghai-Tibet Plateau, hulless barley has a cultivation history of 3500 years, and is the main food, fuel and livestock feed for Tibetans.

Highland barley is mainly distributed in the northwest and southwest provinces of China. It is suitable for growing on plateaus with cool climates, and has the characteristics including strong cold tolerance, short growth period, high yield, early maturity and wide adaptability.

Hulless barley is rich in calcium, iron, vitamins, β-glucan, dietary fiber and other nutrients. For Tibetans, hulless barley grains can be fried and ground into powder, which can be kneaded with buttered tea or green tea into dough for eating, called Tsampa. Hulless barley can also be brewed into hulless barley wine, which is a low-alcohol barley wine.

### SUMMARY

In view of this, the present disclosure provides a black hulless barley extract, which has a function of regulating immunity.

In some embodiments, provided is use of a black hulless barley extract in preparation of a composition for regulating immunity of a subject. The black hulless barley extract is obtained by extracting seeds of black hulless barley (Hordeum vulgare L. var. nudum Hook. f.) with an amylase aqueous solution.

In some embodiments, provided is a method for regulating immunity, including administering to a subject in need thereof a composition including an effective dose of black hulless barley extract. The black hulless barley extract is obtained by extracting seeds of black hulless barley (Hordeum vulgare L. var. nudum Hook. f.) with an amylase aqueous solution.

In some embodiments, the extraction of the foregoing black hulless barley extract is carried out at 80° C. to 100° C. for 50 min to 70 min.

In some embodiments, the foregoing extraction of the black hulless barley extract is carried out at 90° C.±5° C. for 60 min.

In some embodiments, the amylase includes at least one of α-amylase, β-amylase, glucoamylase, and isoamylase.

In some embodiments, a concentration of amylase in the amylase aqueous solution is 0.3% (w/v) to 0.7% (w/v).

In some embodiments, a weight ratio of the amylase aqueous solution to the seeds of the black hulless barley is 5-10:1. In some embodiments, the foregoing weight ratio is 10:1.

In some embodiments, the black hulless barley extract regulates an expression level of an immune-related gene in the subject. In some embodiments, the foregoing immune-related gene is an interleukin-1β (IL-1β) gene, an interleukin-8 (IL-8) gene, an interleukin-6 (IL-6) gene, an interleukin-10 (IL-10) gene, an interferon-γ (IFNG) gene, or any combination thereof.

In some embodiments, the black hulless barley extract enhances a cell killing ability of natural killer cells (NK cells) in the subject.

In some embodiments, regulating the immunity of the subject is to reduce a content of nitric oxide (NO) produced by inflammation.

In some embodiments, the black hulless barley extract reduces immunodeficiency-related symptoms in the subject. In some embodiments, the foregoing immunodeficiency-related symptoms comprise listlessness, gastrointestinal discomfort, split lip, cough, headache, or any combination thereof.

In some embodiments, the black hulless barley extract relieves allergic reactions in the subject. In some embodiments, the foregoing allergic reactions include allergic asthma, food allergy, and skin allergy.

In some embodiments, the black hulless barley extract increases contents of total T cells and helper T cells in blood of the subject.

In some embodiments, the black hulless barley extract increases a CD4/CD8 ratio in blood of the subject.

Based on the above, the black hulless barley extract of the embodiments of the present disclosure is prepared by extracting the seeds of the black hulless barley with the amylase aqueous solution. The black hulless barley extract can be used for preparing the composition for regulating the immunity of the subject. In some embodiments, the black hulless barley extract regulates the expression level of one or more immune-related genes, enhances the cell killing ability of natural killer cells, reduces the content of nitric oxide caused by inflammation, increases the contents of total T cells and helper T cells in blood of the subject, increases the CD4/CD8 ratio in blood of the subject, thereby effectively enhancing overall immunity of the subject, reducing inflammation of the subject, maintaining normal immunity, reducing immunodeficiency-related symptoms (e.g., listlessness, gastrointestinal discomfort, split lip, cough, and headache) of the subject, and relieving allergic reactions (e.g., allergic asthma, food allergy, and skin allergy) of the subject.

### BRIEF DESCRIPTION OF THE DRAWINGS

FIG. 1 is a diagram showing comparative experiment results of a black hulless barley extract and a hulless barley extract with respect to immune-related genes (IL-1β gene and IL-8 gene);

FIG. 2 is a diagram showing comparative experiment results of a black hulless barley extract and a hulless barley extract with respect to immune-related genes (IL-6 gene and IL-10 gene);

FIG. 3 is a diagram showing comparative experiment results of a black hulless barley extract and a hulless barley extract with respect to an immune-related gene (IFNG gene);

FIG. 4 is a diagram showing comparative experiment results of a black hulless barley extract and a hulless barley extract with respect to a cell killing ability of natural killer cells (NK cells);

FIG. 5 is a diagram showing experimental results of a black hulless barley extract with respect to a content of nitric oxide (NO);

FIG. 6 is a diagram showing results of average contents of total T cells in blood of subjects at week 0, week 2 and week 4;

FIG. 7 is a diagram showing results of average contents of helper T cells in blood of subjects at week 0, week 2 and week 4;

FIG. 8 is a diagram showing results of average CD4/CD8 ratios in blood of subjects at week 0, week 2 and week 4;

FIG. 9 is a diagram showing test results of cell killing abilities of natural killer cells (NK cells) in blood of subjects at week 0 and week 4;

FIG. 10 is a diagram showing results of average IgE contents in blood of all subjects at week 0, week 2 and week 4;

FIG. 11 is a diagram showing results of average IgE contents in blood of part of subjects with excessive IgE contents at week 0, week 2 and week 4;

FIG. 12 is a diagram showing survey results of questionnaires on feelings of subjects after administration of the black hulless barley extract for four weeks; and

FIG. 13 is a diagram showing survey results of overall efficacy satisfaction of subjects after administration of the black hulless barley extract for four weeks.

### DETAILED DESCRIPTION

Statistical analysis is performed using software Excel herein. Data are presented as mean ± standard deviation (SD), and differences between groups are analyzed by student's t-test. In the figures, “*” or “#” represents a p-value less than 0.05, “**” or “##” represents a p-value less than 0.01, and “***” or “###” represents a p-value less than 0.001. The more “*” or “#”, the more statistically significant the difference.

The term “extract” refers to the product prepared by extraction. The extract can be present in the form of a solution dissolved in a solvent, or the extract can be present as a concentrate or essence containing no or substantially no solvent, or as a dried powder.

Black hulless barley, whose scientific name is Hordeum vulgare L. var. nudum Hook. f., is commonly known as black barley, black highland barley and the like. Black hulless barley is mature seeds of Hordeum grains in the family Gramineae, named for its dark purple glume and seed epidermis, and is oval or rhombic in shape. Black hulless barley is rich in calcium, iron, vitamins, β-glucan, dietary fiber, trace elements and other nutrients.

In some embodiments, a black hulless barley extract can be obtained by mixing seeds of black hulless barley with an amylase aqueous solution, and then carrying out extraction at a specific temperature for a certain period. The seeds of the black hulless barley are mature seeds, hulled seeds, processed seeds and the like. For example, the seeds of the black hulless barley for extraction are hulled, and then pulverized and ground into a powder.

The amylase aqueous solution is prepared by dissolving amylase in water. In some embodiments, the amylase may be at least one of α-amylase, β-amylase, glucoamylase, and isoamylase or a mixture thereof. In some embodiments, the amylase may be glucoamylase (glucan 1,4-α-glucosidase).

In some embodiments, a concentration of the amylase in the amylase aqueous solution may be 0.3% (w/v) to 0.7% (w/v). For example, the concentration of the amylase is 0.5% (w/v).

In some embodiments, a mixing weight ratio of the seeds of the black hulless barley to the amylase aqueous solution is 5-10:1. For example, the foregoing weight ratio is 10:1. Here, if the amount of the amylase aqueous solution is too small, the extraction efficiency will decrease significantly.

In some embodiments, the extraction of the black hulless barley extract is carried out at 80° C. to 100° C. For example, the extraction is carried out at 85° C. to 95° C. In some embodiments, the extraction of the black hulless barley extract is carried out for 50 min to 70 min. For example, the extraction is carried out for 60 min. In an example, the black hulless barley extract is prepared by extraction at 90° C.±5° C. for 60 min. Here, if the time of the extraction is too short, the extraction efficiency will decrease significantly. If the time of the extraction is too long, degradation of active ingredients in the prepared extract may occur.

In some embodiments, the preparation of the black hulless barley extract further includes filtration, concentration and pH adjustment. For example, the hulled seeds of the black hulless barley are ground into powder, added to a 0.3% (w/v) to 0.7% (w/v) amylase aqueous solution, and extracted at 80° C. to 100° C. for 50 min to 70 min to obtain a first extract liquid of black hulless barley. Next, the first extract liquid of black hulless barley is filtered through a 400-mesh sieve to remove fine solids. Then, the filtered first extract liquid of black hulless barley is concentrated under reduced pressure at 60° C.±5° C. until a Brix value of the solution is 5.0+0.5 degrees Brix, thereby obtaining the black hulless barley extract. If the pH of the prepared black hulless barley extract is higher than 4.0, 0.26% of malic acid may be added to reduce the pH to 3.2+0.5.

In some embodiments, the black hulless barley extract has a function of regulating immunity. For example, regulating the immunity is to regulate an expression level of an immune-related gene, enhance a cell killing ability of natural killer (NK) cells, reduce a content of nitric oxide produced by inflammation, increase an IgE content in blood, increase contents of total T cells and helper T cells in blood, increase a CD4/CD8 ratio in blood, etc. In other words, after being administrated to the subject, the black hulless barley extract can effectively enhance overall immunity, resist inflammation, reduce inflammation, reduce allergic reactions, maintain normal immune function, etc.

In some embodiments, the black hulless barley extract can enhance expression of immune-related genes such as an interleukin-1β (IL-18) gene, an interleukin-8 (IL-8) gene, an interleukin-6 (IL-6) gene and an interleukin-10 (IL-10) gene, and reduce expression of an interferon-γ (IFNG) gene. The protein encoded by the IL-1β gene can activate vascular endothelial cells and lymphocytes, destroy local tissues and make it easier for immune cells to enter. The protein encoded by the IL-8 gene can have chemotaxis on neutrophils and regulate inflammatory response. The protein encoded respectively by the IL-6 gene and the IL-10 gene can enhance the anti-inflammatory ability. The protein encoded by the IFNG gene is a macrophage activating factor. Macrophage activation will lead to inflammatory response. Therefore, by inhibiting the expression of the IFNG gene, the inflammation can be reduced.

In some embodiments, the black hulless barley extract can activate cytotoxicity of NK cells, i.e., enhance the cell killing ability of the NK cells so as to enhance human immunity. The activated NK cells cause apoptosis of target cells, and thus, can kill virus-infected cells, cancer cells, cancerous cells, aged cells or stressed cells.

In some embodiments, the black hulless barley extract can effectively reduce a content of nitric oxide produced by inflammation, so as to reduce the inflammation rate. Macrophages in the human body will promote the production of nitric oxide and cause cell death when they contact pathogens. Therefore, reducing the content of nitric oxide produced by inflammation can reduce inflammation.

In some embodiments, the black hulless barley extract can increase contents of total T cell and helper T cells in blood and increase a CD4/CD8 ratio in blood, so as to enhance immunity. T cells are mainly responsible for cellular immune regulation and secretion of cytokines, and helper T cells are responsible for activating immunoreaction, so increasing the contents of total T cells and helper T cells can enhance immunity. CD4 is the marker on the surface of helper T cells, and CD8 is the marker on the surface of suppressor T cells, so the CD4/CD8 ratio is an important index to observe whether the immune function is normal. Generally speaking, a low CD4/CD8 ratio indicates low immunity, and an increase of the CD4/CD8 ratio indicates an enhancement of the immunity.

In some embodiments, the black hulless barley extract can effectively reduce an IgE content in blood of a subject to relieve allergic reactions. Generally speaking, subjects with recent allergic symptoms or those who are frequently allergic tend to have significantly increased IgE contents in their blood. Therefore, after being administrated to the subject, the black hulless barley extract reduces the IgE content in blood, and can relieve allergic reactions, for example, relieve allergic asthma (e.g., less cough, headache, and other cold symptoms), reduce food allergy (e.g., less gastrointestinal discomfort) and reduce skin allergy (e.g., less split lip or fast healing after split lip).

In some embodiments, the black hulless barley extract reduces immunodeficiency-related symptoms of the subject, such as listlessness, gastrointestinal discomfort, split lip, cough and headache.

In some embodiments, the black hulless barley extract relieves allergic reactions of the subject, such as allergic asthma, food allergy and skin allergy.

In some embodiments, the black hulless barley extract is used in preparation of a composition for regulating immunity of a subject. For example, the composition may be in the form of capsules containing powder of the black hulless barley extract or beverages containing the liquid black hulless barley extract. In some embodiments, a composition includes a container and a black hulless barley extract contained in the container. For example, the black hulless barley extract beverage is a black hulless barley beverage containing the black hulless barley extract in a container.

In some embodiments, the foregoing subject is a human.

In some embodiments, any of the foregoing compositions may be a pharmaceutical. In other words, the pharmaceutical includes an effective content of black hulless barley extract.

In some embodiments, the foregoing pharmaceutical can be manufactured into dosage forms suitable for being enterally, parenterally, orally or topically administered using techniques well known to those skilled in the art.

In some embodiments, the enteral or oral administration dosage forms may be, but not limited to, tablets, troches, lozenges, pills, capsules, dispersible powder or granules, solutions, suspensions, emulsions, syrup, elixir, slurry or the like. In some embodiments, the parenteral or topical administration dosage forms may be, but not limited to, injections, sterile powder, external preparations or the like. In some embodiments, the injections may be administered by subcutaneous injection, intraepidermal injection, intradermal injection or intralesional injection.

In some embodiments, the foregoing pharmaceutical may include a pharmaceutically acceptable carrier widely used in pharmaceutical manufacturing techniques. In some embodiments, the pharmaceutically acceptable carrier may be one or more of the following carriers: a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome and the like. The type and quantity of carrier to be used are within the professional quality and routine skill of those skilled in the art. In some embodiments, the solvent used as a pharmaceutically acceptable carrier may be water, normal saline, phosphate buffered saline (PBS), or an aqueous solution containing alcohol.

In some embodiments, any of the foregoing compositions may be a non-pharmaceutical edible product. In other words, the edible product includes a specific content of black hulless barley extract. In some embodiments, the edible product may be a general food, a health food or a dietary supplement.

In some embodiments, the foregoing edible product may be manufactured into dosage forms suitable for being orally administered using techniques well known to those skilled in the art. In some embodiments, the foregoing general food may be the edible product itself. In some embodiments, the general food may be, but not limited to, beverages, fermented foods, bakery products or seasonings.

In some embodiments, the obtained black hulless barley extract may be further used as a food additive to prepare a food composition containing the black hulless barley extract. Here, the edible product (i.e., food composition) for ingestion by humans and non-human animals can be prepared from any edible material by adding the black hulless barley extract of any example during the preparation of raw materials or by adding the black hulless barley extract of any example during the production of food by conventional methods.

In some embodiments, the black hulless barley extract contained in the foregoing composition containing the black hulless barley extract may be liquid or solid. For example, the solid may be a powder or tablets.

In some embodiments, the composition is used in an amount of 1.2 g/d to 2 g/d of liquid black hulless barley extract.

In some embodiments, the composition is used in an amount of 0.15 g/d to 0.22 g/d of solid black hulless barley extract.

### Example 1: Preparation Method of Black Hulless Barley Extract

First, hulled seeds of black hulless barley (origin: China) were ground into a powder by a pulverizer (SAMPO 1.5 L KJ-SD15G), and added to a 0.5% (w/v) α-amylase (purchased from AB Enzymes; model 10338) aqueous solution. Here, a weight ratio of the 0.5% (w/v) α-amylase aqueous solution to the powder of seeds of black hulless barley was 10:1.

Next, extraction was carried out at 90° C.±5° C. for 60 min to obtain a first extract liquid of black hulless barley, and the first extract liquid of black hulless barley was filtered through a 400-mesh sieve to remove fine solids. Then, the filtered first extract liquid of black hulless barley was concentrated with a concentrator (brand/model: BUCHI-Rotavapor R-100) under reduced pressure at 60° C.±5° C. until a Brix value of the first extract liquid of black hulless barley was 5.0+0.5 degrees Brix, and a pH of the concentrated product was tested. Here, if the pH of the obtained concentrated product was not higher than 4.0, the concentrated product was the black hulless barley extract. If the pH of the obtained concentrated product was higher than 4.0, 0.26% of malic acid could be added to reduce the pH to 3.2+0.5, thereby obtaining the black hulless barley extract.

### Example 2: Preparation Method of Common Hulless Barley Extract (Hereinafter Referred to as White Hulless Barley Extract)

First, hulled seeds of white hulless barley (origin: China) were ground into a powder by a pulverizer (SAMPO 1.5 L KJ-SD15G), and added to a 0.5% (w/v) α-amylase (purchased from AB Enzymes; model 10338) aqueous solution. Here, a weight ratio of the 0.5% (w/v) α-amylase aqueous solution to the powder of seeds of hulless barley was 10:1.

Next, extraction was carried out at 90° C.±5° C. for 60 min to obtain a first extract liquid of hulless barley, and the first extract liquid of hulless barley was filtered through a 400-mesh sieve to remove fine solids. Then, the filtered first extract liquid of hulless barley was concentrated with a concentrator (brand/model: BUCHI-Rotavapor R-100) under reduced pressure at 60° C.±5° C. until a Brix value of the first extract liquid of hulless barley was 5.0+0.5 degrees Brix, and a pH of the concentrated product was tested. Here, if the pH of the obtained concentrated product was not higher than 4.0, the concentrated product was the white hulless barley extract. If the pH of the obtained concentrated product was higher than 4.0, 0.26% of malic acid could be added to reduce the pH to 3.2+0.5, thereby obtaining the white hulless barley extract.

### Example 3: Immune-Related Gene Experiment

Here, the immune-related genes detected were an IL-Iβ (GeneID: 3553) gene, an IL-8 (GeneID: 3576) gene, an IL-6 (GeneID: 3569) gene and an IL-10 (GeneID: 3586) gene.

First, 1.5×10⁵ THP-1 human acute monocytic leukemia (ATCC, TIB202; hereinafter referred to as THP-1 cells) were inoculated into a six-well cell culture plate containing 2 ml of cell culture medium per well, and cultured at 37° C. for 24 h. Here, the cell culture medium used was an RPMI-1640 medium (purchased from Gibco) with 10% fetal bovine serum (purchased from Gibco), 0.05 mM 2-mercaptoethanol, 100 units/mL penicillin and 100 μg/mL streptomycin.

The THP-1 cells were divided into a blank group, a contrast group and an experimental group. The cell culture medium of each group was removed and replaced with 2 mL of experimental medium per well. Then, the culture plate was placed at 37ºC, and the culture was continuously carried out for 48 h. The experimental medium of the experimental group was a cell culture medium containing 0.125 mg/mL black hulless barley extract prepared in Example 1; the experimental medium of the contrast group was a cell culture medium containing 0.125 mg/mL hulless barley extract prepared in Example 2; and the experimental medium of the blank group was a cell culture medium without any extract.

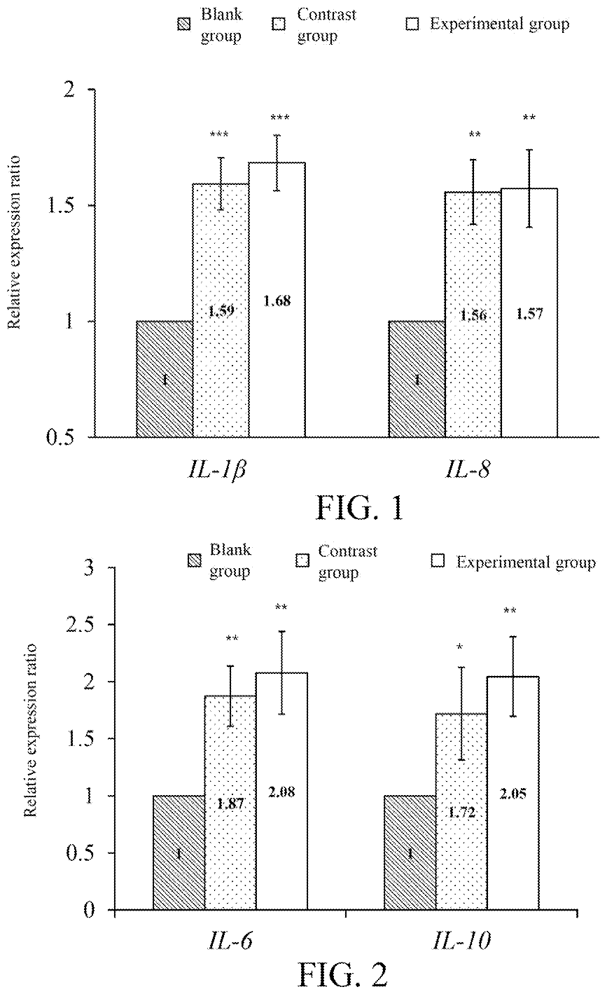

The THP-1 cells of each group were collected, and treated with an RNA extraction reagent kit (purchased from Geneaid, Taiwan, Lot No. FC24015-G) to extract RNA for each group. Next, taking 1000 ng of RNA as a template for each group, the RNA was converted into the corresponding cDNA via reverse transcription by using SuperScript® III reverse transcriptase (purchased from Invitrogene, USA, Catalog No. 18080-051). Then, by using ABI StepOnePlus™ Real-Time PCR system (Thermo Fisher Scientific, USA), KAPA SYBR FAST (purchased from Sigma, USA, Catalog No. 38220000000) and primers (SEQ ID NO: 1 to SEQ ID NO: 8) in Table 1, the cDNA of each group was subjected to a quantitative real-time reverse transcription polymerase chain reaction to observe expression levels of the IL-1δ gene, the IL-8 gene, the IL-6 gene and the IL-10 gene in the THP-1 cells. Conditions set for the instrument of the quantitative real-time reverse transcription polymerase chain reaction were: 95° C. for 20 s, 95° C. for 3 s, and 60° C. for 30 s, for 40 cycles. A 2-ACt method was used for gene quantification, as shown in FIG. 1 and FIG. 2. Here, the quantitative real-time reverse transcription polymerase chain reaction on the cDNA could indirectly quantify the mRNA expression level of the gene, thereby inferring the expression level of the protein encoded by the gene.

It should be particularly noted that the gene expression in FIG. 1 and FIG. 2 was presented in relative expression ratio. The standard deviation was calculated using a STDEV formula in software Excel, and the statistically significant difference between the groups was analyzed by student's t-test. Moreover, “*” is the P value compared with the blank group.

TABLE 1

| Target | Primer | Sequence | | Primer |
|---|---|---|---|---|
| gene | name | number | Sequence | length |
| IL-18 | IL-1β F | SEQ ID | AGCTACGAATCTCC | 20 |
| | | NO: 1 | GACCAC | |
| | IL-1β R | SEQ ID | CGTTATCCCATGTG | 22 |
| | | NO: 2 | TCGAAGAA | |
| IL-8 | IL-8 F | SEQ ID | ACTGAGAGTGATTG | 23 |
| | | NO: 3 | AGAGTGGAC | |
| | IL-8 R | SEQ ID | AACCCTCTGCACCC | 21 |
| | | NO: 4 | AGTTTTC | |
| IL-6 | IL-6 F | SEQ ID | CCTGAACCTTCCAA | 21 |
| | | NO: 5 | AGATGGC | |
| | IL-6 R | SEQ ID | TTCACCAGGCAAGT | 21 |
| | | NO: 6 | CTCCTCA | |
| IL-10 | IL-10 F | SEQ ID | TCAAGGCGCATGTG | 20 |
| | | NO: 7 | AACTCC | |
| | IL-10 R | SEQ ID | GATGTCAAACTCAC | 22 |
| | | NO: 8 | TCATGGCT | |

In Table 1, F is a forward primer, and R is a reverse primer.

Referring to FIG. 1, the relative expression levels of the IL-1β gene and the IL-8 gene of the blank group were regarded as 1.00 (i.e., the expression level of each gene of the blank group was 100%). Compared with the blank group, the relative expression level of the IL-1β gene of the experimental group was 1.68, and the relative expression level of the IL-1β gene of the contrast group was 1.59. The relative expression level of the IL-8 gene of the experimental group was 1.57, and the relative expression level of the IL-8 gene of the contrast group was 1.56. Here, compared with the blank group, the expression level of the IL-Iβ gene of the experimental group was increased by 1.68 times, and the expression level of the IL-8 gene was increased by 1.57 times. In other words, the expression levels of the IL-Iβ gene and the IL-8 gene of the experimental group were significantly increased as compared with the contrast group and the blank group, indicating that the black hulless barley extract could effectively increase the expression levels of the IL-1β gene and the IL-8 gene. In other words, after being administrated to the subject, the black hulless barley extract enhanced the expression levels of the IL-1β gene and the IL-8 gene, thereby activating immune cells, regulating inflammatory response, enhancing the overall immunity and achieving the anti-inflammatory function.

Referring to FIG. 2, the relative expression levels of the IL-6 gene and the IL-10 gene of the blank group were regarded as 1.00 (i.e., the expression level of each gene of the blank group was 100%). Compared with the blank group, the relative expression level of the IL-6 gene of the experimental group was 2.08, and the relative expression level of the IL-6 gene of the contrast group was 1.87. The relative expression level of the IL-10 gene of the experimental group was 2.05, and the relative expression level of the IL-10 gene of the contrast group was 1.72. Here, compared with the blank group, the expression level of the IL-6 gene of the experimental group was increased by 2.08 times, and the expression level of the IL-10 gene was increased by 2.05 times. In other words, the expression levels of the IL-6 gene and the IL-10 gene of the experimental group were significantly increased as compared with the contrast group and the blank group, indicating that the black hulless barley extract could effectively increase the expression levels of the IL-6 gene and the IL-10 gene, and the expression levels of the two genes were significantly increased as compared with those of the contrast group using the hulless barley extract. In other words, after being administrated to the subject, the black hulless barley extract enhanced the expression levels of the IL-6 gene and the IL-10 gene, thereby enhancing the overall immunity and the anti-inflammatory ability.

### Example 4: Immune-Related (Anti-Inflammatory) Gene Experiment

Here, the immune-related (anti-inflammatory) gene detected was an IFNG (GeneID: 3458) gene.

First, 1×10⁶ HaCaT human keratinocytes (ATCC; hereinafter referred to as HaCaT cells) were inoculated into a six-well cell culture plate containing 2 ml of cell culture medium per well, and cultured at 37° C. for 24 h. Here, the cell culture medium used was DMEM (Dulbecco's Modified Eagle Medium; purchased from Gibco) with 10% fetal bovine serum (purchased from Gibco), 100 units/mL penicillin and 100 μg/mL streptomycin.

The HaCaT cells were divided into a blank group, a control group, a contrast group and an experimental group. The cell culture medium of each group was removed and replaced with 2 mL of experimental medium per well. Then, the culture plate was placed at 37° C., and the culture was continuously carried out for 24 h. The experimental medium of the experimental group was a cell culture medium containing 2 μg/ml lipopolysaccharide (LPS; purchased from Sigma, SI-L2880-25MG) and 0.125 mg/mL black hulless barley extract prepared in Example 1; the experimental medium of the contrast group was a cell culture medium containing 2 μg/ml LPS and 0.125 mg/mL hulless barley extract prepared in Example 2; the experimental medium of the control group was a cell culture medium containing 2 μg/ml LPS; and the experimental medium of the blank group was a cell culture medium without any extract or LPS.

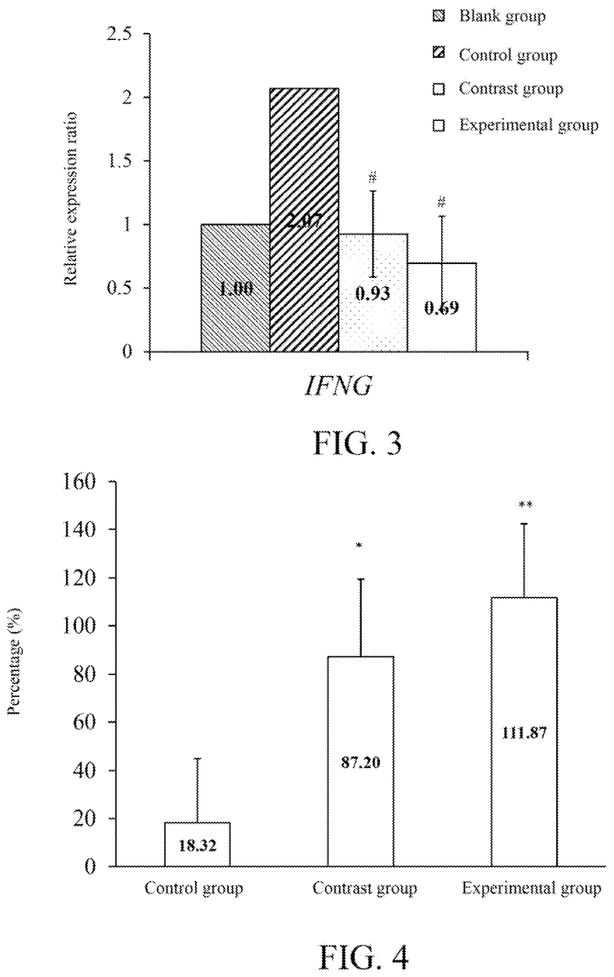

The HaCaT cells of each group were collected, lysed with 500 μL of RB buffer and treated with an RNA extraction reagent kit (purchased from Geneaid, Taiwan, Lot No. FC24015-G) to extract RNA for each group. Next, taking 1000 ng of RNA as a template for each group, the RNA was converted into the corresponding cDNA via reverse transcription by using SuperScript® III reverse transcriptase (purchased from Invitrogene, USA, Catalog No. 18080-051). Then, by using ABI StepOnePlus™ Real-Time PCR system (Thermo Fisher Scientific, USA), KAPA SYBR FAST (purchased from Sigma, USA, Catalog No. 38220000000) and primers (SEQ ID NO: 9 to SEQ ID NO: 10) in Table 2, the cDNA of each group was subjected to a quantitative real-time reverse transcription polymerase chain reaction to observe the expression level of the IFNG gene in the HaCaT cells. Conditions set for the instrument of the quantitative real-time reverse transcription polymerase chain reaction were: 95° C. for 20 s, 95° C. for 3 s, and 60° C. for 30 s, for 40 cycles. A 2-ACt method was used for gene quantification, as shown in FIG. 3. Here, the quantitative real-time reverse transcription polymerase chain reaction on the cDNA could indirectly quantify the mRNA expression level of the gene, thereby inferring the expression level of the protein encoded by the gene.

It should be particularly noted that the gene expression in FIG. 3 was presented in relative expression ratio. The standard deviation was calculated using a STDEV formula in software Excel, and the statistically significant difference between the groups was analyzed by student's t-test. Moreover, “#” is the P value compared with the control group.

TABLE 2

| Target | Primer | Sequence | | Primer |
|---|---|---|---|---|
| gene | name | number | Sequence | length |
| IFNG | IFNG F | SEQ ID | TGCATCGTTTTGGGT | 21 |
| | | NO: 9 | TCTCTT | |
| | IFNG R | SEQ ID | AGTTTGAAGTAAAAG | 27 |
| | | NO: 10 | GAGACAATTTGG | |

In Table 2, F is a forward primer, and R is a reverse primer.

Referring to FIG. 3, the relative expression levels of the IFNG gene of the blank group was regarded as 1.00 (i.e., the expression level of each gene of the blank group was 100%), and the relative expression level of the IFNG gene of the control group as the case of LPS-induced inflammation was 2.07. Compared with the blank group and the control group, the relative expression level of the IFNG gene of the experimental group was 0.69, and the relative expression level of the IFNG gene of the contrast group was 0.93. Here, both the experimental group and the contrast group could inhibit the expression of the IFNG gene, and the black hulless barley extract of the experimental group could significantly inhibit the IFNG gene. In other words, the expression level of the IFNG gene of the experimental group was significantly reduced as compared with the control group, the blank group and the contrast group, indicating that the black hulless barley extract could effectively inhibit the expression level of the IFNG gene. In other words, after being administrated to the subject, the black hulless barley extract could enhance the expression level of the IFNG gene, thereby reducing the probability of macrophage activation, enhancing the overall immunity and reducing inflammation.

### Example 5: Cell Killing Ability Experiment of Killer Cells

The fluorescent dye 3,8-diamino-5-[3-(diethylmethylammonio)propyl]-6-phenylphenanthridinium diiodide (propidium iodide, PI) is a nuclear staining reagent that can stain DNA, and it can emit red fluorescence after being intercalated into double-stranded DNA. Since PI cannot pass through the living cell membrane, but can pass through the damaged cell membrane to stain its nucleus, PI can be used as a nucleic acid reagent for dead cells. PI is often used for detecting cell apoptosis. PI is also often used with calcein acetoxymethyl ester (calcein AM) or fluorescein diacetate (FDA) or other fluorescent probes to stain living cells and dead cells at the same time.

Here, human peripheral blood mononuclear cells (PBMC; hereinafter referred to as PBMC cells) (containing about 10% natural killer cells) and K562 human myeloid leukemia cells (purchased from American Type Culture Collection (ATCC), Catalog No. CCL-243; hereinafter referred to as K562 cells) were co-cultured, and the number of dead cells was analyzed using the dead cell stain PI in combination with flow cytometry, thereby indirectly inferring the cell killing ability of the natural killer cells of each group.

First, peripheral blood was provided by healthy donors, and PBMC cells were isolated from the peripheral blood. 6 mL of venous blood was collected from each of several subjects using a purple-top blood collection tube containing an anticoagulant EDTA (purchased from Greiner Bio-One, Catalog No. 455036). Next, the collected venous blood was centrifuged at a speed of 300×g for 15 min, and 2 mL of leukocytic cream of buffy coat was taken. The leukocytic cream was diluted with 2 mL of DPBS into 4 mL. Next, the diluted leukocytic cream was slowly added to a centrifuge tube containing 3 mL of Ficoll-Paque Plus (purchased from GE Healthcare, Catalog No. 17144002). During the addition, the diluted leukocytic cream and the Ficoll-Paque Plus should be in separated layers and not mixed. Next, the centrifuge tube containing the diluted leukocytic cream and the Ficoll-Paque Plus in separated layers was centrifuged at 400×g for 40 min, and the supernatant was removed. 2 mL to 3 mL of PBMC cells were taken from the intermediate layer in the centrifuge tube after centrifugation, rinsed with 3 times by volume of 1X DPBS, and uniformly mixed with the foregoing 1X DPBS buffer to form a PBMC mixture. Next, the PBMC mixture was centrifuged at a speed of 300×g for 10 min to form a supernatant and a PBMC cell pellet.

The PBMC cell pellet was redissolved with 5 mL of an RPMI 1640 cell culture medium (purchased from Gibco, Catalog No. 31800022), 1 μl of calcein AM (volume ratio of dyeing 1:5000; purchased from Invitrogen, Catalog No. A13201) was added, and the mixture was allowed to stand for 5 min for staining. Next, the PBMC cells were centrifuged at a speed of 400×g for 5 min to remove the supernatant, followed by the addition of 5 mL 1×PBS, and the centrifugation was repeated for 3 to 5 times to ensure the removal of the excess calcein AM.

A RPMI 1640 cell culture medium was used to dilute the PBMC cells into 1×10⁶/100 μl, and the K562 cells into 5×10⁴/100 μl. Next, the stained and diluted PBMC cells were inoculated into a V-bottom 96-well plate at 1×10⁶ PBMC cells per well. Next, the cells of the blank group, the control group, the contrast group and the experimental group were respectively prepared according to Table 3. In the 0 h group and the 6 h group of the control group, the contrast group and the experimental group, 5×10⁴ K562 cells per well were added to the V-bottom 96-well plate containing the stained PBMC cells in each group. In addition, the blank group was divided into a PBMC cell group, a K562 cell group, and a K562 cell and alcohol (EtOH) group. In the PBMC cell group of the blank group, only 1×10⁶ PBMC cells were added, and the RPMI 1640 cell culture medium was added to a volume of 200 μl per well; in the K562 cell group of the blank group, only 5×10⁴ K562 cells were added, and the RPMI 1640 cell culture medium was added to a volume of 200 μl per well; and in the K562 cell and alcohol (EtOH) group of the blank group, 5×10⁴ K562 cells and 95% alcohol were added, and the RPMI 1640 cell culture medium was added to a volume of 200 μl per well.

TABLE 3

| | Control | Contrast | Experimental | Blank |
|---|---|---|---|---|
| | group | group | group | group |
| 0 h | PBMC cells/ | PBMC cells/ | PBMC cells/ | PBMC cells |
| | K562 cells | K562 cells | K562 cells | K562 cells |
| 6 h | PBMC cells/ | PBMC cells/ | PBMC cells/ | K562 |
| | K562 cells | K562 cells | K562 cells | cells+95% |
| | | | | alcohol |

Next, the sample to be tested (the hulless barley extract prepared in Example 2 and the black hulless barley extract prepared in Example 1) was added in the contrast group and the experimental group, and no additional sample to be tested was added in the control group. 0.125 mg/mL hulless barley extract prepared in Example 2 was added in the 0 h group and the 6 h group of the contrast group. 0.125 mg/mL black hulless barley extract prepared in Example 1 was added in the 0 h group and the 6 h group of the experimental group.

Immediately after the addition of the sample to be tested, 1 μL of PI stain (propidium iodide, purchased from BD Biosciences, Catalog No. 556463; volume ratio 1:200) was added in the 0 h groups of the control group, the contrast group and the experimental group, and the three groups of the blank group.

The three groups of the blank group were transferred to a flow cytometry (purchased from BD, Model Accuri™ C6 Plus) to detect distribution of scatterplots. Populations of PBMC and K562 were boxed in the scatterplots for signal quantification of red fluorescence (PE-A) in the subsequent experiment. Next, PE-A fluorescence signals of the 0 h groups of the control group, the contrast group and the experimental group were detected.

6 h after the addition of the sample to be tested, 1 μL of PI stain was added to the 6 h groups of the control group, the contrast group and the experimental group, and the PE-A fluorescence signals were immediately detected by the flow cytometry.

Next, negative gate and positive gate were boxed in the PE-A fluorescence histogram. The proportion of dead cells in total cells could be calculated based on the positive gate. Thereby, the percentage of red positive gate of each well could be compared, and the killing ability of NK cells could be expressed as a percentage of number of dead cells/number of protocells. The experimental results are shown in FIG. 4.

Referring to FIG. 4, the percentage of the killing ability of NK cells of the control group was 18.32%, the percentage of the killing ability of NK cells of the contrast group was 87.20%, and the percentage of the killing ability of NK cells of the experimental group was 111.82%, indicating that the killing ability of NK cells of the experimental group was enhanced by 6 times or so. In other words, after being administrated to the subject, the black hulless barley extract could enhance the killing ability of NK cells, thereby enhancing the immunity of the subject.

### Example 6: Anti-inflammatory experiment

First, 1×10⁴ mouse macrophages (RAW 264.7; ATCC TIB-71; hereinafter referred to as RAW cells) were inoculated into a 96-well cell culture plate containing 200 μL of cell culture medium per well, and cultured at 37° C. in 5% CO2 for 24 h. Here, the cell culture medium used was a DEME medium (purchased from Gibco) with 10% fetal bovine serum (purchased from Gibco), 1% penicillin-streptomycin (purchased from Gibco, Catalog No. 15140122) and 4 mM L-glutamine (purchased from Gibco).

The RAW cells were divided into a blank group, a control group and an experimental group. The cell culture medium of each group was removed and replaced with 200 μL of experimental medium per well. Then, the culture plate was placed at 37° C., and the culture was continuously carried out for 24 h. The experimental medium of the experimental group was a DMEM medium containing 500 ng/ml lipopolysaccharide (LPS; purchased from Sigma, SI-L2880-25MG), 0.125 mg/mL black hulless barley extract prepared in Example 1, 1% AA and 4 mM L-glutamine; the experimental medium of the control group was a DMEM medium containing 2 μg/ml LPS, 1% AA and 4 mM L-glutamine; and the experimental medium of the blank group was a DEME medium containing 1% AA and 4 mM L-glutamine.

150 μL of the RAW cells of each group and the experimental medium thereof after the reaction were taken out and added to a 96-well plate containing 130 μL of secondary water per well. A Griess reagent (reagent A: reagent B=1:1) was prepared using a Griess reagent kit (Life technologies; 1445263). 20 μL of Griess reagent was respectively added to the foregoing 96-well plate, and the reaction was carried out in the dark for 30 min.

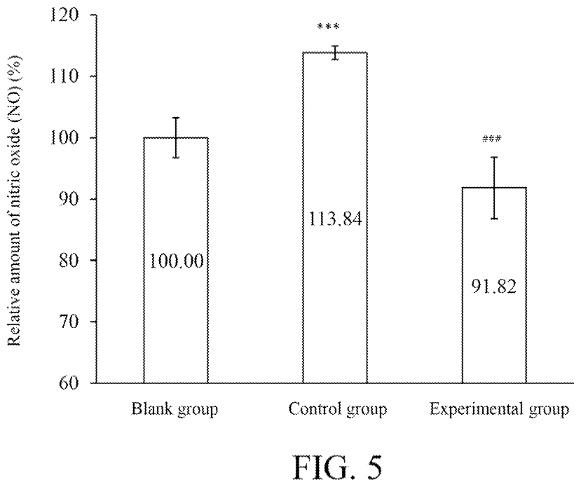

Next, after the completion of the reaction, the absorbance was read at a wavelength of 548 nm using an ELISA immunoassay analyzer. A larger reading of the OD value indicates a higher content and concentration of NO. Moreover, the NO value here is an inflammatory index. It should be particularly noted that the relative amount of NO in FIG. 5 was presented in percentage. The standard deviation was calculated using a STDEV formula in software Excel, and the statistically significant difference between the groups was analyzed by student's t-test. In FIG. 5, “*” is the P value compared with the blank group, and “#” is the P value compared with the control group.

Referring to FIG. 5, the relative amount of NO of the blank group was 100%, and the relative amount of NO of the control group was 113.84%, indicating that the LPS could stimulate the inflammatory response of RAW cells in deed. The relative amount of NO of the experimental group was 91.82%, indicating that the black hulless barley extract could effectively reduce the content of NO in deed in the case of LPS-induced inflammation. Moreover, the relative amount of NO of the experimental group was lower than the relative amount of NO of the blank group, and much lower than the relative amount of NO of the control group. Thereby, after being administrated to the subject, the black hulless barley extract could effectively reduce the content of NO and reduce the inflammation rate.

### Example 7: Human Experiment

Herein, 2 g of black hulless barley extract prepared in Example 1 was mixed with 48 g of water to prepare 50 mL of black hulless barley extract beverage.

Subjects: 10 subjects. The subjects were healthy adults aged from 20 to 50, who felt themselves as immunodeficiency.

Test items: Proportions of total T cells and helper T cells in blood (immunity index), CD4/CD8 ratio in blood (immunity index), killing ability of NK cells in blood, IgE concentration in blood (allergy index), and self-evaluation questionnaire (immunodeficiency-related symptoms).

Test method: 50 mL of black hulless barley extract beverage was administrated to the 10 subjects daily for four consecutive weeks. Before administration (i.e., week 0), after 2 weeks of administration (i.e., week 2) and after 4 weeks of administration (i.e., week 4), 30 mL of blood was respectively collected from each subject and tested for its immunity indexes, allergy index and killing ability of NK cells, and the immunodeficiency-related symptoms of the subjects were evaluated by self-evaluation questionnaires. For the proportions of total T cells and helper T cells in blood, the CD4/CD8 ratio in blood, and the IgE concentration in blood, the blood tests were conducted by LeZen Reference Lab. The tests of the killing ability of NK cells in blood were conducted by TCI GENE INC.

It should be particularly noted that in the accompanying drawings, “*” means that the p value is less than 0.05 compared with week 0, “**” means that the p value is less than 0.01 compared with week 0, and “***” means that the p value is less than 0.001 compared with week 0.

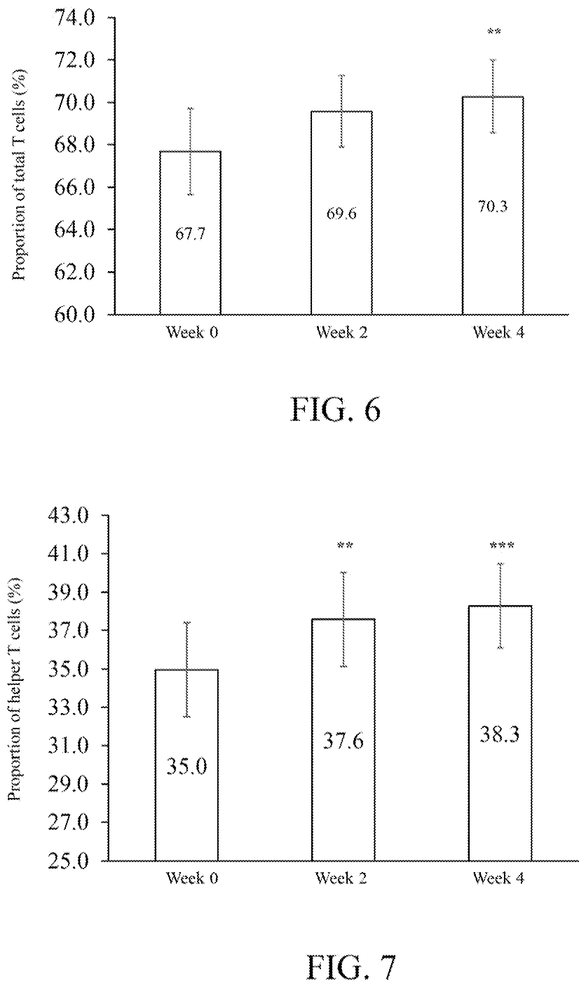

Referring to FIG. 6, at week 0, the average proportion of total T cells in blood of the 10 subjects was 67.7%. After the administration of the black hulless barley extract beverage for 2 weeks, the average proportion of total T cells in blood of the 10 subjects was increased to 69.6%, which was an increase of about 2.8%. After the administration of the black hulless barley extract beverage for 4 weeks, the average proportion of total T cells in blood of the 10 subjects was increased to 70.3% as compared with week 0, which was an increase of about 3.8%. As can be seen, after being administrated to the subjects, the beverage containing the black hulless barley extract increased the proportion of total T cells in blood, thereby regulating the body cellular immunity and secretion of cytokines, and improving the immunity.

Referring to FIG. 7, at week 0, the average proportion of helper T cells in blood of the 10 subjects was 35.0%. After the administration of the black hulless barley extract beverage for 2 weeks, the average proportion of helper T cells in blood of the 10 subjects was increased to 37.6%, which was an increase of about 7.4%. After the administration of the black hulless barley extract beverage for 4 weeks, the average proportion of helper T cells in blood of the 10 subjects was increased to 38.3% as compared with week 0, which was an increase of about 9.4%. As can be seen, after being administrated to the subjects, the beverage containing the black hulless barley extract increased the proportion of helper T cells in blood, thereby activating the immunoreaction and enhancing the immunity.

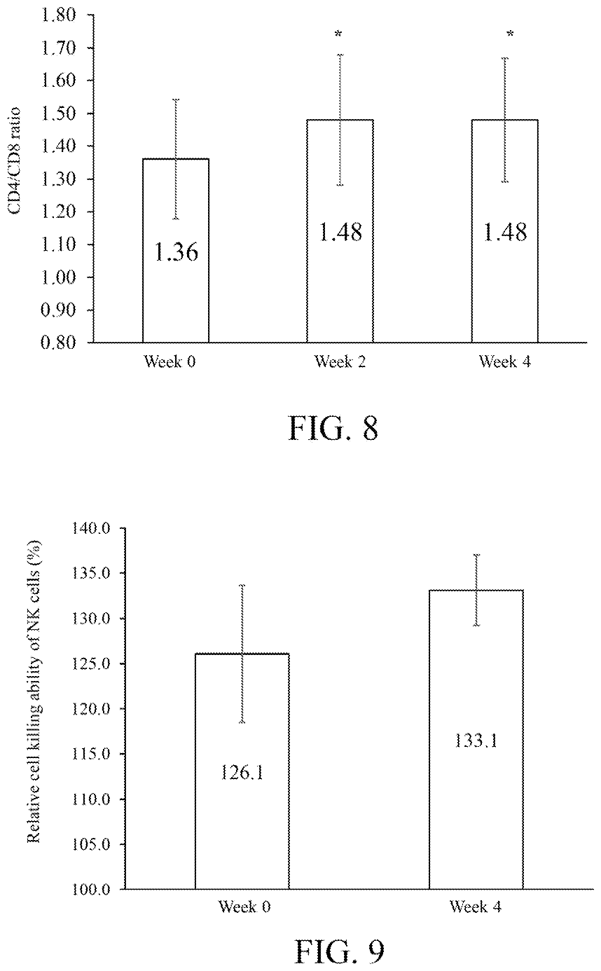

Referring to FIG. 8, CD4 is the marker on the surface of helper T cells, and CD8 is the marker on the surface of suppressor T cells, so the CD4/CD8 ratio is an important index to observe whether the immune function is normal. At week 0, the average CD4/CD8 ratio in blood of the 10 subjects was 1.36. After the administration of the black hulless barley extract beverage for 2 and 4 weeks, the average CD4/CD8 ratios in blood of the 10 subjects were both 1.48. As can be seen, after the beverage containing the black hulless barley extract was administrated to the subjects for 2 weeks, the average CD4/CD8 ratio in blood could be increased, indicating a normal immune function of the subjects.

Referring to FIG. 9, at week 0, the average killing ability of NK cells in blood of the 8 subjects was 126.1%. After the administration of the black hulless barley extract beverage for 4 weeks, the average killing ability of NK cells in blood of the 8 subjects was enhanced by 7% to reach 133.1%. As can be seen, after the beverage containing the black hulless barley extract was administrated to the subjects, the cell killing ability of NK cells in blood could be enhanced. After the killing ability of NK cells stress was enhanced, the NK cells could kill not only cancer cells, but also virus-infected cells, cancerous cells, and aged or stressed cells, and thereby, the overall immunity of the subjects could be enhanced.

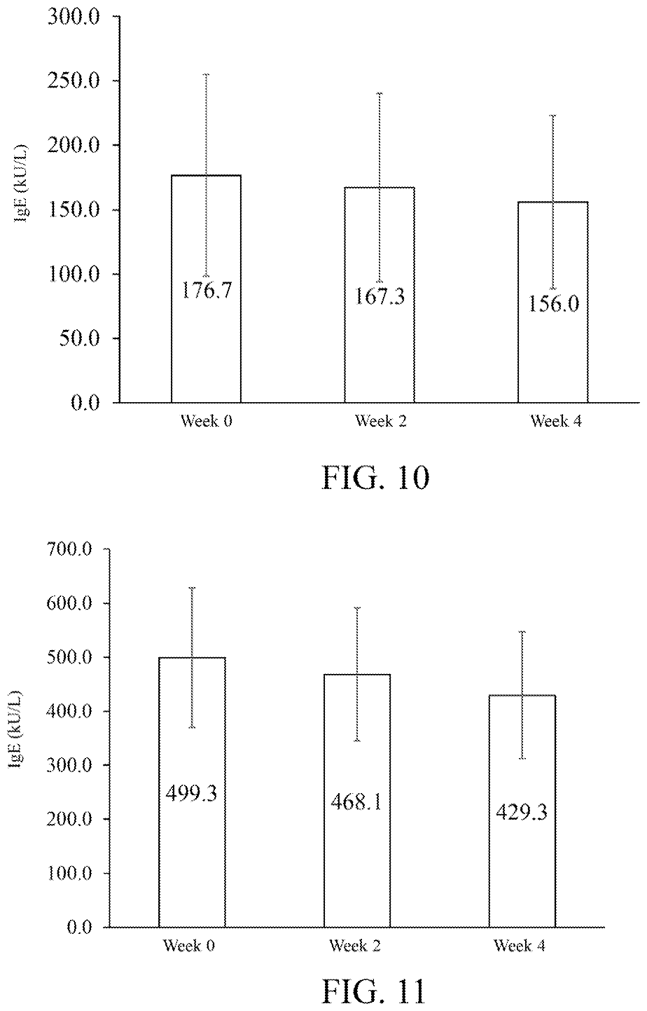

Referring to FIG. 10, at week 0, the average IgE concentration in blood of the 10 subjects was 176.7 kU/L. After the administration of the black hulless barley extract beverage for 2 weeks, the average IgE concentration in blood of the 10 subjects was reduced to 167.3 kU/L. After the administration of the black hulless barley extract beverage for 4 weeks, the average IgE concentration in blood of the 10 subjects was reduced to 156.0 kU/L. In other words, at week 2, the average IgE concentration in blood of the 10 subjects was reduced by 5.3%, and at week 4, the average IgE concentration in blood of the 10 subjects was reduced by 11.7%. As can be seen, after the beverage containing the black hulless barley extract was administrated to the subjects, the average IgE concentration in blood could be reduced, indicating a relief of allergic reactions of the subjects.

Referring to FIG. 11, 3 of the 10 subjects had an excessive IgE concentration, so the changes of the IgE concentration of the 3 subjects were further observed. At week 0, the average IgE concentration in blood of the 3 subjects was 499.3 kU/L. After the administration of the black hulless barley extract beverage for 2 weeks, the average IgE concentration in blood of the 3 subjects was reduced to 468.1 kU/L. After the administration of the black hulless barley extract beverage for 4 weeks, the average IgE concentration in blood of the 3 subjects was reduced to 429.3 kU/L. In other words, at week 2, the average IgE concentration in blood of the 3 subjects was reduced by 6.3%, and at week 4, the average IgE concentration in blood of the 3 subjects was reduced by 14.0%. As can be seen, after the beverage containing the black hulless barley extract was administrated to the subjects, the average IgE concentration in blood could be reduced, indicating a relief of allergic reactions of the subjects.

The 10 subjects respectively filled in the self-evaluation questionnaire at week 0, week 2 and week 4, including overall mental state, gastrointestinal discomfort, split lip, and cold symptoms such as cough and headache. The subjects evaluated their feelings according to the options, which were converted into percentages according to the answers. The evaluation and results of the self-evaluation questionnaire are shown in Table 4.

TABLE 4
1. After administration, feel less cough,
headache, and other mild cold symptoms

| | |
|---|---|
| Strongly feel (3) | 3 subjects |
| Slightly feel (2) | 6 subjects |
| Hardly feel (1) | 1 subject |

2. After administration, feel less split
lip or fast healing after split lip

| | |
|---|---|
| Strongly feel (3) | 1 subject |
| Slightly feel (2) | 8 subjects |
| Hardly feel (1) | 1 subject |

3. After administration, feel less
gastrointestinal discomfort

| | |
|---|---|
| Strongly feel (3) | 5 subjects |
| Slightly feel (2) | 3 subjects |
| Hardly feel (1) | 2 subjects |

4. After administration, feel an enhancement of
overall metal state and less prone to fatigue

| | |
|---|---|
| Strongly feel (3) | 3 subjects |
| Slightly feel (2) | 4 subjects |
| Hardly feel (1) | 3 subjects |

5. Overall efficacy satisfaction

| | |
|---|---|
| Very satisfied (4) | 1 subject |
| Satisfied (3) | 6 subjects |
| Generally satisfied (2) | 3 subjects |
| Not satisfied (1) | 0 subject |

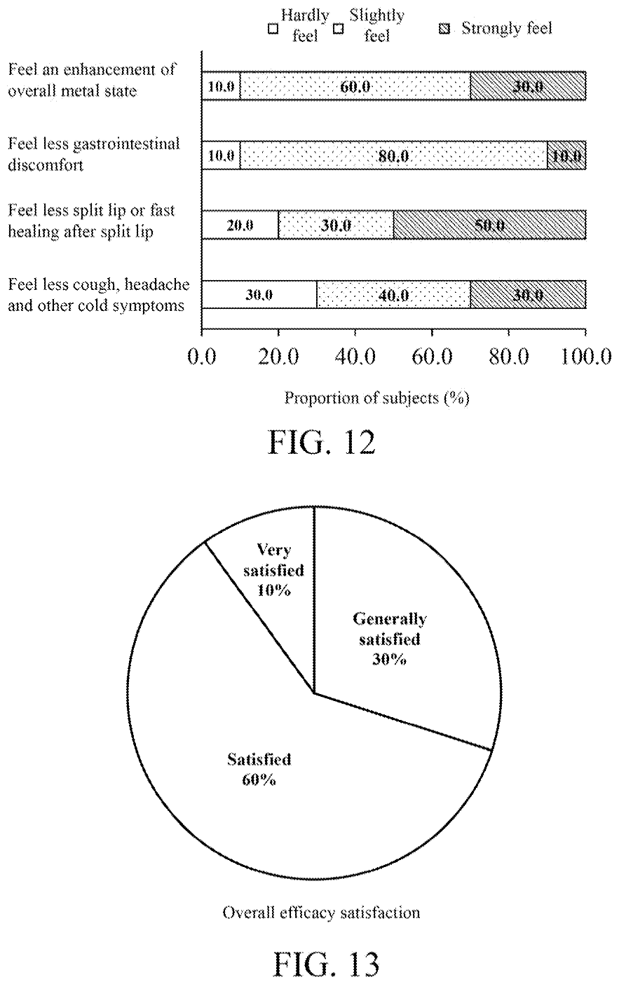

Referring to FIG. 12, after the black hulless barley extract beverage was administrated to the 10 subjects for 4 weeks, for “After administration, feel less cough, headache, and other mild cold symptoms”, 30% (3) of the subjects chose “Strongly feel”, 60% (6) of the subjects chose “Slightly feel”, and 10% (1) of the subjects chose “Hardly feel”.

For “After administration, feel less split lip or fast healing after split lip”, 10% (1) of the subjects chose “Strongly feel”, 80% (8) of the subjects chose “Slightly feel”, and 10% (1) of the subjects chose “Hardly feel”.

For “After administration, feel less gastrointestinal discomfort”, 50% (5) of the subjects chose “Strongly feel”, 30% (3) of the subjects chose “Slightly feel”, and 20% (2) of the subjects chose “Hardly feel”.

For “After administration, feel an enhancement of overall metal state and less prone to fatigue”, 30% (3) of the subjects chose “Strongly feel”, 40% (4) of the subjects chose “Slightly feel”, and 30% (3) of the subjects chose “Hardly feel”.

Referring to FIG. 13, after the black hulless barley extract beverage was administrated to the 10 subjects for 4 weeks, for “Overall efficacy satisfaction”, 10% (1) of the subjects chose “Very satisfied”, 60% (6) of the subjects chose “Satisfied”, 30% (3) of the subjects chose “Generally satisfied”, and 0% (0) of the subjects chose “Not satisfied”.

As can be seen, the black hulless barley extract could improve the immunodeficiency-related symptoms, and the allergic reactions such as allergic asthma, food allergy and skin allergy of the subjects.

Based on the above, the black hulless barley extract according to the embodiments of the present disclosure can be used in the preparation of the composition for regulating the immunity of the subject. The black hulless barley extract is prepared by extracting the seeds of the black hulless barley with the α-amylase aqueous solution. In some embodiments, the black hulless barley extract has the functions such as regulating the expression level of the immune-related gene, enhancing the cell killing ability of the natural killer cells, reducing the content of nitric oxide caused by inflammation, increasing the contents of total T cells and helper T cells in the blood of the subject, inhibiting the IgE content in the blood of the subject, and increasing the CD4/CD8 ratio in blood of the subject, and can enhance overall immunity of the subject, reduce inflammation of the subject, maintain normal immunity, reduce immunodeficiency-related symptoms (e.g., listlessness, gastrointestinal discomfort, split lip, cough, and headache) of the subject, and relieve allergic reactions (e.g., allergic asthma, food allergy, and skin allergy) of the subject.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A method for regulating immunity, comprising administering to a subject in need thereof a composition comprising an effective dose of black hulless barley extract, wherein the black hulless barley extract is obtained by extracting seeds of black hulless barley (Hordeum vulgare L. var. nudum Hook. f.) with an amylase aqueous solution.

2. The method according to claim 1, wherein the extraction is carried out at 80° C. to 100° C. for 50 min to 70 min.

3. The method according to claim 2, wherein the extraction is carried out at 90° C.±5° C. for 60 min.

4. The method according to claim 1, wherein the amylase aqueous solution comprises at least one of α-amylase, β-amylase, glucoamylase, and isoamylase.

5. The method according to claim 1, wherein a concentration of amylase in the amylase aqueous solution is 0.3% (w/v) to 0.7% (w/v).

6. The method according to claim 1, wherein a weight ratio of the amylase aqueous solution to the seeds of the black hulless barley is 5-10:1.

7. The method according to claim 6, wherein the weight ratio is 10:1.

8. The method according to claim 1, wherein the black hulless barley extract regulates an expression level of an immune-related gene in the subject.

9. The method according to claim 8, wherein the immune-related gene is an interleukin-1β (IL-1β) gene, an interleukin-8 (IL-8) gene, an interleukin-6 (IL-6) gene, an interleukin-10 (IL-10) gene, an interferon-γ (IFNG) gene, or any combination thereof.

10. The method according to claim 1, wherein the black hulless barley extract enhances a cell killing ability of natural killer cells (NK cells) in the subject.

11. The method according to claim 1, wherein the black hulless barley extract reduces a content of nitric oxide (NO) caused by inflammation in the subject.

12. The method according to claim 1, wherein the black hulless barley extract reduces immunodeficiency-related symptoms in the subject.

13. The method according to claim 12, wherein the immunodeficiency-related symptoms comprise listlessness, gastrointestinal discomfort, split lip, cough, headache, or any combination thereof.

14. The method according to claim 1, wherein the black hulless barley extract relieves allergic reactions in the subject.

15. The method according to claim 14, wherein the allergic reactions comprise allergic asthma, food allergy, and skin allergy.

16. The method according to claim 1, wherein the black hulless barley extract increases contents of total T cells and helper T cells in blood of the subject.

17. The method according to claim 1, wherein the black hulless barley extract increases a CD4/CD8 ratio in blood of the subject.
